(19) United States
(12) Patent Application Publication
(10) Pub. No.: US 2012/0065670 A1
(43) Pub. Date: Mar. 15, 2012

(54) DEVICE FOR SEALING AN INCISION

(75) Inventors: Hans-Jürgen Tiedtke, Bonn (DE); Ralf Hornig, Bonn (DE); Gisbert Richard, Hamburg (DE)

(73) Assignee: IMI INTELLIGENT MEDICAL IMPLANTS AG, Zug (CH)

(21) Appl. No.: 13/255,880

(22) PCT Filed: Mar. 10, 2009
(86) PCT No.: PCT/EP2009/001708

§ 371 Date: Nov. 15, 2011

(51) Int. Cl. A61B 17/03

(52) U.S. Cl. 606/213

(57) ABSTRACT

The present invention provides a plug device or grommet (1) for sealing an incision in tissue of a human or animal body, such as an incision in the tissue of the sclera (S) made during a surgical intervention for the implantation of a visual prosthesis. The device (1) comprises: a cover part (10) for covering the incision and an area of the tissue (S) around the incision; and an insertion part (20) extending from the cover part (10) for insertion into the incision through the tissue (S). The insertion part (20) defines a sealable passage (30) which is adapted to extend through the incision, and the insertion part (20) is preferably flexible and resiliency deformable to open and close the sealable passage (30) there-through. The sealable passage (30) extends through the incision to provide communication between an outer side (S₀) of the tissue and an inner side (Sᵢ) of the tissue. The passage (30) may accommodate a component which provides a connection or interconnection through the tissue (S).

### TECHNICAL FIELD

The present invention relates to a device and method for sealing an incision made in tissue of a human or animal body during a surgical intervention. In particular, the device and method of the present invention have application in closure of incisions where a surgical implant requires a physical connection through the incision.

In one particular application, for example, the device of the invention functions as a plug or grommet and is especially suitable for use with an ocular implant which is introduced and extends through an incision made in the sclera into the posterior chamber of the eye. Accordingly, the device of the invention may be embodied as a plug device or a closure device for sealing an incision through the sclera (i.e. a sclerotomy) and it will be convenient to hereinafter describe the invention in this exemplary context. It will be appreciated, however, that the present invention is not limited to this particular application, but may be suitable for use in a wide variety of surgical interventions.

### BACKGROUND OF THE INVENTION

In recent years, several systems have been developed for intraocular drug delivery and retinal stimulation that involve piercing the sclera for access to either the posterior chamber or the anterior chamber of the eye. The region of the sclera typically selected as the site for incision is the region known as the pars plana. Where the systems involve implanted devices, part of which is required to be within the orbit of the eye and part of which remains outside the eyeball, it is often the case that a physical connection through the tissue of the sclera is required on a medium- or long-term basis. Although a physical connection between components of an implanted device can be achieved and maintained through the tissue of the sclera, this does present particular challenges and/or difficulties to be managed during the post-surgical care of the patient.

In the surgical procedure for the implantation of a visual prosthesis device which comprises, for example, an epiretinal implant, an incision is typically made to form a small flap in the wall of the sclera, and this is then peeled back for insertion of the implant components. During this procedure, an amount of the vitreous humour present in the posterior chamber of the eye may be removed and replaced with a saline solution, which over a period of time will usually be naturally substituted with aqueous humour produced in the anterior chamber of the eye. After the implantation is complete, the flap formed in the sclera is folded back and closed with sutures around a thin, insulated electrical cable, which then connects the epiretinal implant within the eye to extra-ocular electronic control elements.

If a significant amount of the relatively high viscosity, gel-like vitreous humour is replaced by the considerably lower viscosity saline solution and/or aqueous humour, the potential for leakage of fluid from the eye and an associated loss of pressure may become a consideration during the procedure and while the incision through the sclera is healing. Furthermore, as the incision through the sclera heals, scar tissue typically forms at the site of the incision through which the electrical cable extends. This scar tissue can make removal and reinsertion of an implant more complicated in the event that replacement of the visual prosthesis device becomes necessary. In other words, not only is a further incision generally required, but the scar tissue on or around the cable can complicate its removal and an excessive amount of scar tissue may develop in this particular region of the sclera.

While it has been suggested to coat the parts of the implant that extend through the sclera with a bio-compatible soft polymer material in order to support and accelerate the healing process, this alone does not solve the problems associated with the formation of scar tissue outlined above. Furthermore, this approach can render a subsequent explantation of the device more complicated due to scar tissue adhering to the soft polymer material. It also does not address the management of fluid leakage from the eye.

The international patent application published as WO 2008/109862 A2 describes a clamp-type sclerotomy grommet comprising an upper half and a lower half joined by a hinge and being openable and closable via a snap-type closure opposite the hinge. This clamp-type grommet is designed to be placed on an electrical cable of a retina implant for location in an incision in the sclera (i.e. a sclerotomy) to improve the sclerotomy seal, to locate the electrical cable in the incision and to minimize torque applied to the retina by the electrode array. The rounded shape of the grommet is intended to provide an improved seal for the incision. A significant disadvantage of this arrangement, however, is that the grommet is not designed to be re-opened after it has been deployed and the incision through the sclera has largely healed. Removing the grommet to re-access the cable and/or recover the implanted components thus necessarily involves inflicting further trauma or damage on the tissues of the sclera. Accordingly, although the clamp-type hinged grommet of this prior art arrangement may provide a satisfactory sclerotomy seal, it nevertheless has significant drawbacks.

It would therefore be desirable to provide a device and/or a method for sealing an incision made in tissue of the human or animal body, and in particular in the sclera, which allows for repeated removal and/or re-insertion of an implant without further injury to the sclera tissue, but which nevertheless provides a satisfactory seal that minimizes or prevents fluid loss from the body, such as from an internal chamber of the eye.

### SUMMARY OF THE INVENTION

The present invention provides a device, such as a surgical plug or grommet, for closing or sealing an incision in tissue of a human or animal body, as defined in the independent claims. The invention also provides a method of closing or sealing an incision in tissue of a human or animal body. Preferred features of the invention are recited in the dependent claims.

According to one aspect, therefore, the present invention provides a device, such as a surgical plug or a grommet, for sealing an incision in tissue of a human or animal body, comprising: a cover part for covering the incision and an area of tissue around the incision; and an insertion part which extends from the cover part for insertion into the incision through the tissue; wherein the insertion part defines a sealable passage which is adapted to extend through the incision. The passage is adapted to accommodate a component that provides a connection or interconnection through the tissue.

The plug device or grommet of the present invention is thus designed to provide communication via the sealable passage between an outer side of the tissue and an inner side of the tissue. Where the tissue constitutes, for example, the wall of an organ (e.g. the sclera forms a wall of the eyeball), the device of the invention is able to provide a sealable passage through the incision which facilitates communication between an outer side of the wall of the organ and an inner side of the wall. The device of the invention is therefore designed to remain in the patient after the surgical procedure is completed to provide the capacity for a medium- to long-term connection via the sealable passage.

In a preferred form of the invention, the cover part of the device comprises a surface which is adapted to contact the area of the tissue around the incision; that is, on an outer side of the tissue in which the incision is made. Preferably, the cover part has a relatively low profile, in the sense that it does not project to a large or significant extent on the outer side of the tissue. In particular, the cover part is desirably formed to have a generally flat configuration. In a preferred embodiment, for example, the cover part comprises a flange member for engaging and seating against the tissue surrounding the incision. Accordingly, the flange member presents a surface which is adapted to contact and seat against the area of the tissue around the incision.

In a preferred form of the invention, the insertion part of the device has an extent or dimension in the direction through the incision (i.e. substantially perpendicular to a plane of the outer surface of the tissue) which approximately corresponds to or is slightly greater than a thickness of the tissue itself. For example, where the tissue comprises a wall of an organ, the insertion part is desirably sized to extend from the cover part a distance which is approximately equal to or slightly greater than the thickness of the wall of the organ. In a particularly preferred embodiment, the insertion part projects somewhat beyond the inner side of the tissue, e.g. beyond an inner wall of the organ.

In a preferred form of the invention, the sealable passage extends through the insertion part and is separated or isolated from the incision in the tissue by the material of the insertion part. Accordingly, the passage provides a channel or path through the incision and yet may prevent contact with the incised tissue itself.

In one particular form of the invention, the passage extends or opens laterally through a side portion of the insertion part, such that the passage is not only axially accessible (i.e. from either end of the insertion part) but is also laterally accessible (i.e. from the side). This lateral access can substantially simplify the accommodation of an elongate component within the passage, as the component need not be threaded through one of the ends of the passage, but rather can be slotted into the passage from the side. After a component has been accommodated within the passage, e.g. via the side or lateral access, the lateral portions of the insertion part providing that access can then be fixed together (e.g. with sutures) to isolate the component from contact with the incised tissue.

In a preferred form of the invention, the sealable passage is arranged generally centrally through the insertion part of the device and extends in a direction generally perpendicular to the surface of the tissue in which the incision is made. Moreover, the sealable passage also preferably extends through the cover part of the device, again preferably in a central region thereof. In an alternative form of the invention, however, the sealable passage may provide an angled or curved path through the insertion part and, thus, through the incision. In this regard, the passage can be designed to be angled or curved to match a desired path or configuration of the component to be accommodated in the passage.

In a preferred form of the invention, the insertion part is flexible and resiliently deformable to open and close the sealable passage there-through. For example, the insertion part preferably comprises at least one flexible member which at least partially defines the passage, and the at least one flexible member is resiliently deformable to open and close said passage. More preferably, the insertion part comprises a pair of opposed flexible members which together at least partially define the sealable passage, and the pair of flexible members are resiliently deformable to open and close said passage.

The insertion part of the plug-like device or grommet of the invention, and in particular the at least one flexible member of the insertion part, is preferably biased to close or seal the passage which extends there-through. In this way, the passage will tend to close and seal automatically against and/or around a component accommodated therein. In this respect, the resiliently deformable member(s) of the insertion part may be inherently biased to a closed or sealed position. Alternatively, a separate biasing means (e.g. a spring means) may be provided to urge or bias the insertion part, and in particular the at least one flexible member of the insertion part, to close or seal the passage. Such a separate biasing means may be located in and/or extend around the insertion part of the device.

In a preferred form of the invention, the device further comprises attachment means for securely attaching the device to the area of the tissue around the incision. In one particular embodiment, the attachment means comprises an attachment member which extends laterally from the cover part. For example, the attachment member may comprise a flexible web of material. This flexible web may be an integrally formed extension of the cover part or it may a separate member, which is attached to the cover part. The flexible web may be designed to be fixed by sutures or tacks to the tissue surrounding the incision thereby to attach the device securely in position. Alternatively, or in addition, the attachment means may comprise an adhesive for adhesively fixing the cover part to the area of tissue surrounding the incision. In a particular embodiment, for example, a surface of the cover part adapted for contact and engagement with the tissue may comprise an adhesive layer which, when brought into contact with the tissue, securely bonds or affixes the cover part—and therewith the entire device—to the tissue around the incision. In another particular embodiment, the flexible web of material may comprise an adhesive layer which, when brought into contact with the tissue, securely bonds or affixes the web and therewith the cover part of the device to the tissue around the incision.

In a preferred form of the invention, the sealable passage in the device is adapted to accommodate at least one component which, in use, is intended to extend through the tissue. For example, the passage may be shaped to conform with a surface profile of the at least one component it is intended to accommodate. In this way, the passage in the closed state may be adapted to seal against an outer surface or outer profile of the component it accommodates. In a particularly preferred embodiment of the invention, the passage is intended to accommodate an elongate element having a substantially uniform cross-section. The passage thus preferably presents a corresponding uniform cross-section. In the case of a retinal implant, for example, the at least one component intended to be accommodated in the passage of the device is a cable providing electrical communication between components of the implant. The sealable passage through the insertion part of the device thus preferably has a cross-section which, in the closed state, is adapted to seal against an outer surface of the cable. The cable may be a substantially flat, ribbon-type cable and the passage may therefore, for example, be formed as a slot for accommodating the ribbon cable. By forming the sealable passage with a curved configuration, as noted above, sharp angles or “kinks” in the path of the ribbon cable through the plug or grommet device of the invention can thereby be avoided.

In a preferred form of the invention, the device is fabricated from a bio-compatible polymer material, such as a polyamide, a parlyene or a silicone polymer. In this regard, silicone polymers are particularly preferred, not only for their bio-compatible characteristics, but also because they exhibit the desired degree of softness and elasticity. The device (e.g. plug or grommet) of the invention is preferably formed as an integral (i.e. unitary) element. In other words, the cover part and the insertion part may be integrally formed from a soft, flexible, bio-compatible polymer material. According to another aspect, the present invention provides a method of closing or sealing an incision in tissue of a human or animal body, comprising the steps of:
providing a device having a cover part for covering the incision and an area of the tissue around the incision, and an insertion part extending from the cover part for insertion into the incision through the tissue, wherein the insertion part defines a sealable passage; and
inserting the device into the incision in such a manner that the cover part covers the incision and an area of the tissue around the incision, that the insertion part extends into the incision through the tissue, and that the sealable passage extends through the incision to provide communication between an outer side of the tissue and an inner side of the tissue.

In a preferred form of the invention, the method further includes the step of attaching the device to the area of the tissue around the incision. The step of attaching the device to the area of the tissue around the incision may include adhering or bonding the cover part to the tissue. For example, the step of attaching the device to the area of the tissue around the incision may comprise adhesively bonding a flexible web of material connected to the cover part to the tissue. Alternatively, the step of attaching the device to the area of the tissue around the incision may comprise attaching such a flexible web of material to the tissue with sutures and/or tacks.

### BRIEF DESCRIPTION OF THE DRAWINGS

The above and further features and advantages of the invention will become more readily apparent from the following detailed description of preferred embodiments of the invention with reference to the accompanying drawings, in which like reference characters identify like features, and in which:

FIG. 1 is a sectioned side view of a device according to a preferred embodiment of the invention shown in use, deployed through an incision in the tissue of the sclera;

FIG. 2 is a bottom-end view of the device shown in FIG. 1, with a ribbon-cable of a retina implant shown extending from the bottom or lower end of the device;

FIG. 3 is a sectioned side view of a device according to another embodiment of the invention, shown in use, deployed through an incision in the tissue of the sclera;

FIG. 4 is a sectioned side view of a device according to a further embodiment of the invention, shown in use deployed through an incision in the tissue of the sclera;

FIG. 5 is a sectioned side view of a device according to yet another embodiment of the invention, shown in use, deployed through an incision in the tissue of the sclera;

FIG. 6 is a top view of the device shown in FIG. 5;

FIG. 7 is a sectioned side view of a device according to another embodiment of the invention shown in use, and largely corresponds to FIG. 1;

FIG. 8 is a bottom-end view of the device shown in FIG. 7, with a ribbon-cable of a retina implant shown extending from the bottom or lower end of the device.

### DETAILED DESCRIPTION OF THE PREFERRED EMBODIMENTS

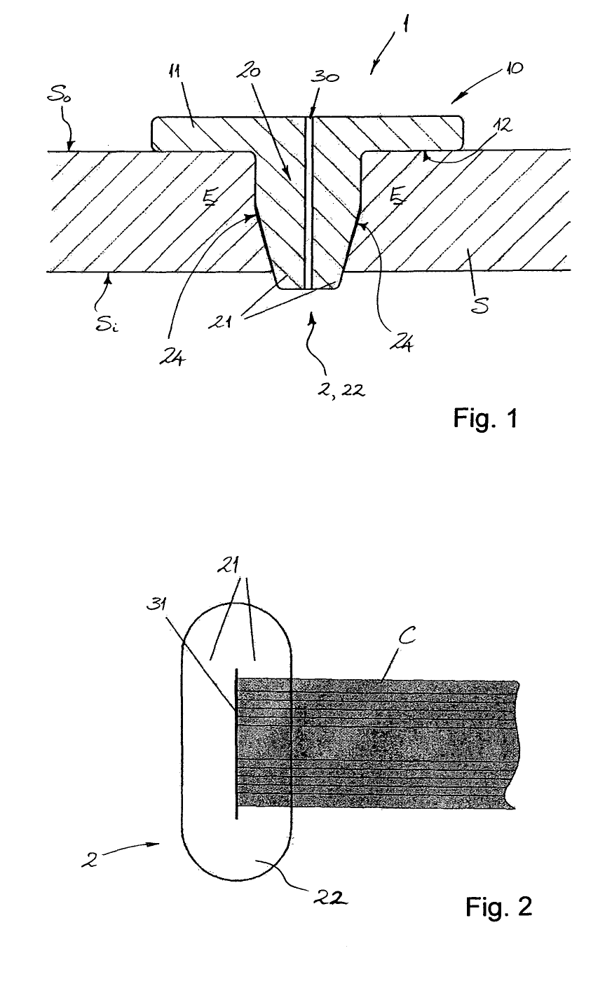

With reference to FIG. 1 of the drawings, a device 1 for covering or sealing an incision made in tissue of a human or animal body during a surgical intervention is illustrated in a deployed or in-use state, penetrating the tissue wall of the sclera S of a patient. The device 1 is in the form of a plug or grommet and comprises a cover part 10 which is for covering the incision and an area of the tissue surrounding the incision. In this respect, the cover part 10 comprises a flange member 11 having a lower surface 12 which is adapted to engage and seat against the tissue on an outer side Sₒ of the sclera S around the incision.

The plug-like device 1 furthermore incorporates an insertion part 20 which extends generally centrally from the cover part 10 and is adapted for insertion into the incision through the tissue of the sclera S. The insertion part 20 comprises a pair of opposed flexible members 21, between which a passage 30 in the form of a slot is defined extending between an upper surface 13 of the cover part 10 and a distal or inner end 22 of the opposed flexible members 21. In other words, the elongate slot forming the passage 30 extends through a central region of the cover part 10 and between the opposed flexible members 21 of the insertion part 20 to provide communication between the external or outer side Sₒ and the internal or inner side Sᵢ of the sclera S.

Importantly, the opposed flexible members 21 are resiliently deformable to effect opening and closing of the passage 30. In other words, the flexible members 21 which at least partially define the passage 30 there-between are able to function in the form of a valve. That is, when they are moved towards one another or pressed together (e.g. under an inherent or external bias), the opposed flexible members 21 close and thereby seal the passage 30 through the sclera. When they are moved apart from one another, the flexible members 21 operate to open the passage 30. In this manner, the sealing plug device 1 of the present invention is able to effect a closing and re-opening of the aperture or passage 30 formed through the sclera S without imparting further trauma to the tissue itself.

With reference now to FIG. 2 of the drawings, the distal or inner end 2 of the plug device or grommet 1 shown in FIG. 1 is illustrated in an in-use condition, with a ribbon-cable C of a retina implant extending through the slot-like passage 30. The pair of opposed flexible members 21 are typically resiliently biased towards one another, such that the passage 30 through the device is generally biased into a closed state. However, if the plug device 1 of the invention is appropriately sized with respect to the incision made through the sclera S, the edges E of the incised tissue through which the insertion part 20 of the device extends can in any case bear upon outer surfaces 24 of the flexible members 21 and tend to press those members together, which itself also tends to cause the passage 30 to close. As can therefore be seen in FIG. 2 of the drawings, the slot-like passage 30 at the inner end 2 of the plug-like device 1 is illustrated with the flexible members 21 being squeezed together such that the slot is compressed into a closed slit 31, which seals around the flat ribbon cable C extending there-through.

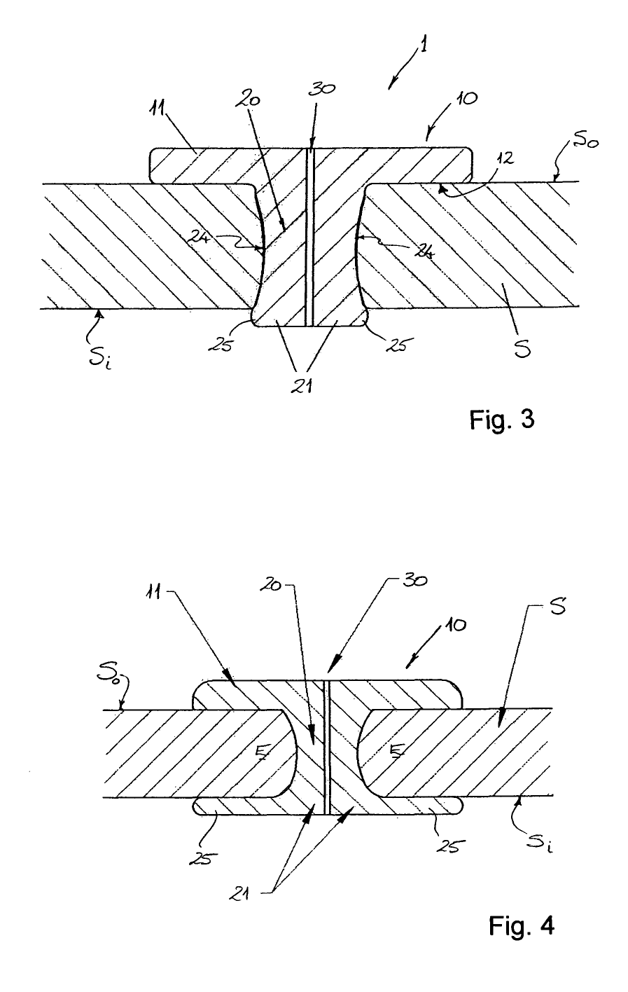

FIGS. 3 and 4 of the drawings illustrate two alternative embodiments of a plug-like device or grommet 1 according to the invention. The main difference between these two embodiments and the embodiment of FIG. 1 is that the distal or inner ends 22 of the pair of opposed flexible members 21 of the insertion part 20 at the inner side Sᵢ of the sclera S diverge from one another to varying degrees. In the embodiment shown in FIG. 1, by contrast, the flexible members 21 taper towards one another to provide the grommet or plug device 1 with an increasingly narrow profile in this region. By flaring or diverging the distal or inner ends 22 of the flexible members 21 away from one another, the device 1 of the invention provides for a better engagement with the tissue at the inner side Sᵢ of the sclera S and thereby achieves a better retention in the surgical incision. Thus, a distal or inner end region 25 of the insertion part 20 is adapted to engage the inner side Sᵢ of the tissue for retaining the plug or grommet 1 in the incision.

The embodiment in FIG. 4 of the drawings provides a more extreme example of this feature than the embodiment of FIG. 3. In particular, in the embodiment of FIG. 4, the device 1 of the invention includes a flange-like structure 25 at the inner side Sᵢ of the sclera S. In other words, the distal ends 22 of the two flexible members 21 remote from the cover part 10 incorporate a flange member 25 for engaging and seating against the inner surface Sᵢ of the tissue of the sclera S in an area around the incision. This structure naturally provides the plug-like device 1 of the invention with extremely good retention properties.

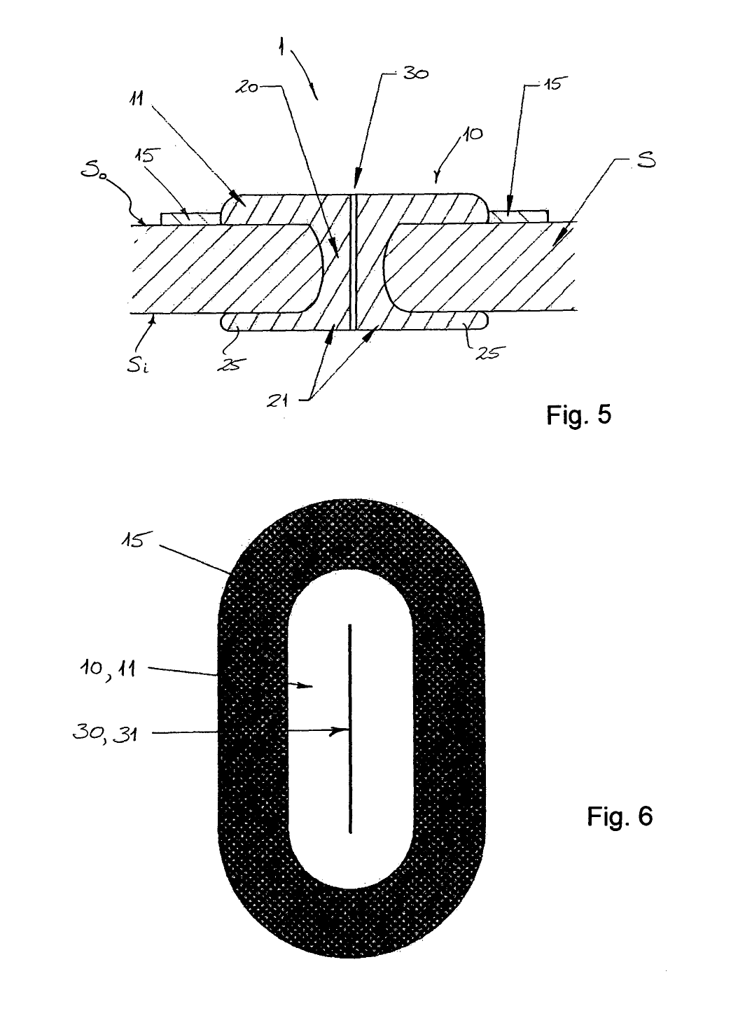

Yet a further embodiment of the present invention is illustrated in FIGS. 5 and 6 of the drawings. Though this particular embodiment has the same general configuration as the embodiment shown in FIG. 4, it further incorporates a web-like member 15 which extends from the outer periphery of the flange member 11 of the cover part 10 for use in securely attaching the device 1 to an area of the tissue surrounding the incision. In this regard, the web 15 of material may be in the form of a film (e.g. a polymer film) or a woven fabric and may serve for the attachment of sutures or fastening tacks (not shown). Alternatively and/or in addition, this web 15 and/or the underside of the flange member 11 of the cover part 10 may incorporate an adhesive layer (e.g. using a bio-compatible adhesive, such as a fibrin adhesive) for securely bonding the plug-like device 1 to an outer surface Sₒ of the sclera tissue.

As return electrodes (not shown) of a retina implant device may be applied to the tissue of the sclera S at the outer surface Sₒ of the eyeball, the web-like member 15 shown in FIGS. 5 and 6 is preferably electrically conductive to avoid any interference with the function of such return electrodes in the external part of the implant.

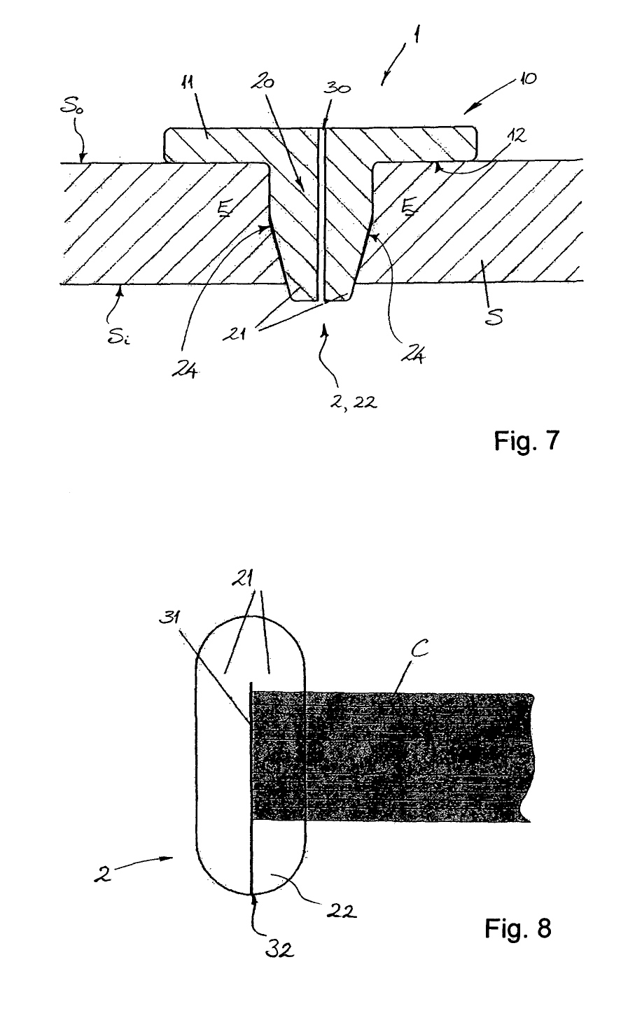

The drawing FIGS. 7 and 8 illustrate a further embodiment of the invention which is the same in most respects as the embodiment shown in drawing FIGS. 1 and 2. In this case, however, the slot-like passage 30 through the insertion part 20 of the device 1 extends laterally to provide a side opening 32 for accessing the passage 30. As the plug-like device or grommet 1 of the invention is desirably formed of a relatively soft and flexible polymer material, it is possible to deform the flexible members 21 apart from one another at the lateral opening 32 to enable insertion of the ribbon cable C into the sealable passage 30 from the side.

The dimensions of the plug-like sealing device 1 of the invention are essentially dictated by the field of its application. In the case of the embodiments illustrated in FIGS. 1 to 8 of the drawings, where the plug-type device 1 is specifically designed for incisions in the sclera associated with retinal implants, the incision will typically be about 6-7 mm in length and the thickness of the sclera at the site of the pars plana is typically about 1-2 mm. Accordingly, the cover-part 10 of the device 1 will typically have a length of in the range of about 8-10 mm and a width of about 5-6 mm and a height perpendicular to the outer surface Sₒ of the sclera of about 1 mm or less. The insertion part 20, on the other hand, will typically have a length corresponding to the length of the incision in the range of about 5-7 mm, a width in the range of about 1-4 mm, and an extent or height through the tissue in the range of about 2-3 mm.

Prior to implantation, the plug device 1 of the invention is desirably threaded onto the ribbon cable C of the retinal implant to be deployed in the eye of the patient. Accordingly, the ribbon cable C may be threaded through the slot-like passage 30 which extends through the cover part 10 and the insertion part 20 of the device 1 before the electrode array of the retinal implant is actually surgically implanted into the eye of the patient. The arrangement of the plug or grommet 1 on the cable C prior to the surgical implantation of the visual prosthesis avoids the complication of the threading procedure being conducted in the course of surgery. With the embodiment of FIGS. 7 and 8, the insertion part provides lateral access to the slot-like passage 30 such that the opposed flexible members 21 may be moved apart to open the slot and insert the ribbon cable from the side. This avoids the need to thread the cable from one of the ends and further simplifies the deployment procedure.

With the plug-like device or grommet 1 positioned such that the ribbon cable C is accommodated within the passage 30, the surgeon may then make the appropriate incision through the sclera in the region of the pars plana and carry out the implantation of retinal electrode array of the implant. Once the electrode array is correctly positioned and affixed to the retina, the plug-like device 1 (which is located on the ribbon cable C and is still external to the eyeball) can be slid along the cable extending through the incision such that the insertion part 20 of the plug is inserted into the incision through the tissue. In this respect, the insertion part 20 is inserted into the incision to the extent that the flange member 11 of the cover part 10 comes into engagement and abutment with the outer surface Sₒ of the sclera S. In this position, the inner surface 13 of the flange member 11 and/or a dependent flexible web 15 is securely attached or fastened to the outer surface Sₒ of the sclera S by adhesive, tacks or suturing, as described above.

In this position, any inner flange member 25 at a distal or inner end 22 of the insertion part 20 should also be in engagement with an inner side S, of the sclera. The opposed flexible members 21 of insertion part 20 either under their own self-bias and/or the action of the edges E of the incised tissue are pressed against the ribbon cable C into a closed condition, thereby to seal the passage 30 against leakage of fluid from the eye.

1. A surgical plug for sealing an incision in tissue of a human or animal body, comprising:
a cover part for covering the incision and an area of the tissue around the incision; and
an insertion part extending from the cover part for insertion into the incision through the tissue;
wherein the insertion part defines a sealable passage which is adapted to extend through the incision.

2. The surgical plug of claim 1, wherein the insertion part is flexible and resiliently deformable to open and close the sealable passage there-through.

3. The surgical plug of claim 2, wherein the insertion part comprises at least one flexible member which at least partially defines the passage, and wherein the flexible member is resiliently deformable to open and close said passage.

4. The surgical plug of claim 3, wherein the insertion part comprises a pair of opposed flexible members which together at least partially define the sealable passage, and wherein the pair of flexible members are resiliently deformable to open and close said passage.

5. The surgical plug of claim 1, wherein the sealable passage extends through i) a central region of the cover part, ii) a central region of the insertion part, or iii) a lateral or side portion of the insertion part.

6-7. (canceled)

8. The surgical plug of claim 1, wherein the cover part comprises a flange member for engaging and seating against the tissue around the incision.

9. The surgical plug of claim 1, further comprising attachment means for securely attaching the device to the area of the tissue around the incision.

10. The surgical plug of claim 7, wherein the attachment means comprises an attachment member extending laterally from the cover part, the attachment member preferably comprising a flexible web.

11. The surgical plug of claim 7, wherein the attachment means comprises an adhesive layer for adhesively fixing the cover part to the area of tissue around the incision.

12. The surgical plug of claim 1, wherein the cover part and the insertion part are integrally formed from a flexible polymer material.

13. A grommet for sealing an incision in tissue of a human or animal body, comprising:
a cover part for covering the incision and an area of the tissue around the incision; and
an insertion part extending from the cover part for insertion into the incision through the tissue;
wherein the insertion part defines a sealable passage which is adapted to extend through the incision for accommodating a component that provides a connection or interconnection through the tissue.

14. The grommet of claim 13, wherein the insertion part is flexible and resiliently deformable to open and close the sealable passage there-through, the insertion part preferably comprising at least one flexible member which at least partially defines the passage and is resiliently deformable to open and close said passage.

15. The grommet of claim 14, wherein the insertion part comprises a pair of opposed flexible members which together at least partially define the sealable passage, and wherein the pair of flexible members are resiliently deformable to open and close said passage.

16. The grommet of claim 13, wherein the sealable passage extends i) through a central region of the cover part, ii) centrally through the insertion part, or iii) through a lateral or side portion of the insertion part.

17-18. (canceled)

19. The grommet of claim 13, wherein the cover part comprises a flange member for engaging and seating against the tissue around the incision.

20. A method of sealing an incision in tissue of a human or animal body, comprising the steps of:
providing a surgical plug device having: a cover part for covering the incision and an area of the tissue around the incision, and an insertion part extending from the cover part for insertion into the incision through the tissue, wherein the insertion part defines a sealable passage; and
inserting the device into the incision in such a manner that the cover part covers the incision and an area of the tissue around the incision, that the insertion part extends into the incision through the tissue and that the sealable passage extends through the incision to provide communication between an outer side of the tissue and an inner side of the tissue.

21. The method of claim 20, further comprising the step of:
attaching the device to the area of the tissue around the incision.

22. The method of claim 21, wherein the step of attaching the device to the area of the tissue around the incision comprises i) adhesively bonding the cover part to the tissue, or ii) adhesively bonding a flexible web connected to the cover part to the tissue.

23. (canceled)

24. The method of claim 23, wherein the step of attaching the device to the area of the tissue around the incision comprises attaching a flexible web connected to the cover part to the tissue with sutures and/or tacks.

25. The method of claim 20, further comprising the step of:
accommodating a component in the sealable passage which provides a connection or interconnection through the tissue.
